(19) United States
(12) Patent Application Publication
(10) Pub. No.: US 2018/0056086 A1
(43) Pub. Date: Mar. 1, 2018

(54) PERCUTANEOUS MODIFICATION OF VASCULAR EXTRACELLULAR MATRIX TO PREVENT AND TREAT VASCULAR RESTENOSIS

(71) Applicant: MOR RESEARCH APPLICATIONS LTD., Tel Aviv (IL)

(72) Inventor: Emanuel HARARI, Kfar HaOranim (IL)

(21) Appl. No.: 15/558,588

(22) PCT Filed: Mar. 17, 2016
(86) PCT No.: PCT/IL2016/050296

§ 371 Date: Sep. 15, 2017

Related U.S. Application Data
(60) Provisional application No. 62/134,796, filed on Mar. 18, 2015.

(51) Int. Cl. A61N 5/06; A61B 17/22; A61M 25/10

(52) U.S. Cl. CPC A61N 5/062; A61B 17/22031; A61M 25/104; A61N 2005/067; A61N 2005/0661; A61N 2005/0659; A61N 2005/0662; A61N 2005/0651

(57) ABSTRACT

Aspects of disclosed embodiments relate to systems, devices and methods for treating and preventing restenosis by cross-linking collagen fibrils of the vessel wall at the intervention site. The devices described are drug-eluting devices comprising: an expandable member or a balloon; a cross-linking agent; and a photoactivating light source. Upon the inflation of the expandable member or the balloon the cross linking agent is released and photoactivated in a therapeutically effective amount for cross-linking collagen fibrils.

### CROSS-REFERENCE TO RELATED APPLICATIONS

This application claims the benefit of U.S. Provisional Patent Application No. 62/134,796 filed Mar. 18, 2015, the contents of which are incorporated herein by reference in their entirety.

### FIELD OF INVENTION

The invention is directed to the field of vascular implants.

### BACKGROUND OF THE INVENTION

Percutaneous transluminal angioplasty (PTA) is a well-established procedure that is used to treat blockages, stenosis, thrombosis and other lesions of blood vessels such as ruptured plaques. During the procedure a catheter is used to advance a balloon to the vascular lesion. Once located in the desired position within the lesion, the balloon is inflated against the vascular wall and the balloon dilates to a predetermined diameter using relatively high pressure of fluid. By doing so, the balloon exerts radial forces, which compress the atherosclerotic plaque. After deflation of the balloon and its withdrawal blood flow is restored through the dilated vessel. The main drawback of this method of intervention the high rate of restenosis, necessitating a repeat procedure or an alternative method of revascularization such as bypass surgery. In order to decrease the risk of restenosis, the angioplasty can be combined with the delivery of a vascular stent. A vascular stent is a tubular endovascular implant which can be composed of a variety of materials: metals such as stainless steel, alloys such as Ni—Ti (Nitinol) and/or biodegradable polymers. The vascular stent is delivered to the lesion through a catheter and deployed by self-expansion or a balloon based delivery system. Once deployed, the stent prevent the elastic recoil of the vessel, maintaining a wider lumen compared to the use of a balloon and significantly reduces the risk of restenosis. Nevertheless, restenosis rate is still high particularly in diabetic patients. In the case of coronary stents the main mechanism of restenosis is the proliferation and migration of smooth muscle cells from the vessel wall to the lumen of the stent forming a layer of neointima, which eventually obstruct the lumen. Reduction of the neointima formation can be achieved by various methods such as brachytherapy and the use of drug-eluting stents. Drug eluting stents (DES) are stents coated with one or several polymers that serve as a delivery system to pharmaceutical active compounds that inhibit the proliferation of smooth muscle cells and reduce the magnitude of neointima formation thereby decreasing and delaying the need for future revascularization. The main drawback of DES compared to bare metal stents (BMS) is that the pharmaceutical active compounds used (e.g., sirolimus, paclitaxel, everolimus, zotarolimus) delay also the endothelization of the implant, exposing it to blood flow and thereby increasing the risk for stent thrombosis. In order to decrease the risk of stent thrombosis the manufacturers and the medical association recommend the use of dual-antiplatelet therapy (DAPT) with both aspirin and P2Y antagonist for a period of 12 month as opposed to 1 to 3 months in the case of BMS. The use of long-term DAPT reduces the risk of stent thrombosis at the expense of increasing the risk of bleeding. In clinical practice this means that surgical procedures should be postponed till it is safe to stop DAPT.

The drug-eluting balloon (DEB) has emerged as a treatment modality for in-stent restenosis. The advantage of this technique is that the drug (e.g., the antiproliferative compound paclitaxel) is administered to the vessel wall without the use of a stent coated with a biostable polymer as a platform for delivery. As a result, the acute neointimal and vascular injury from the procedure is not prolonged by persistent exposure to the drug-carrier platform, which may generate a persistent inflammatory and immunologic reaction. This probably enables shorter period of DAPT in comparison to the use of DES.

Another problem of vascular stenting is distal embolization of particles released from brittle atherosclerotic plaques and thrombus when a balloon is inflated in the vessel and more commonly when a stent is placed. This phenomena described in the coronary circulation is associated with less favorable clinical outcome during percutaneous revascularization. A technology that would stabilize the fibrous element of the plaque will also decrease the burden of distal embolization.

### SUMMARY OF THE INVENTION

The present invention provides devices and methods for treating and preventing restenosis at an intervention site.

In an embodiment, the device disclosed herein comprises an expandable member having a distal end, a proximal end and a working length there between; a cross-linking agent; and a photoactivating light source operative to emit light for activating, when in an operable position, the cross-linking agent to cross-link collagen surrounding the expandable member.

In another embodiment, the expandable member is a balloon.

In one embodiment, the expandable member is coated with a layer comprising a carrying agent. In one embodiment, the expandable member is coated with a layer comprising a carrying agent and a cross linking and/or a cross linkable agent.

In one embodiment, the device further comprises a power source for expanding (e.g., inflating) the expandable member and/or emitting photoactivating light.

In one embodiment, the device further comprises a catheter. In one embodiment, the device comprising a catheter further comprises a guide-wire. In one embodiment, the cross-linking agent is supplied through the catheter. In one embodiment, the photoactivating light source is supplied by and/or incorporated on the expandable member. In one embodiment, the photoactivating light source is incorporated on the guide-wire and/or catheter.

In one embodiment, the cross-linking agent is embedded in the carrying agent layer coating the working length of the expandable member and/or the balloon's exterior.

In one embodiment, the cross-linking agent induces cross-linking of collagen (e.g., collagen fibrils) following activation through a photoactivating light source.

In one embodiment, the cross-linking agent comprises one or more photoactive substances. In one embodiment, the photoactive substance or substances comprise one or more photoactive bioflavonoids selected from the group consisting of: proanthocyanidin, catechin, epicatechin, epigallo catechin, epicatechin gallate, epigallocatechin gallate, quercetin, tannic acid, and any combination thereof.

In one embodiment, the cross-linking agent comprises a photoactive substance that comprises riboflavin.

In one embodiment, the cross-linking agent comprises an enzyme capable of cross-linking collagen fibers.

In one embodiment, the cross-linking agent further comprises a metalloproteinase inhibitor.

In one embodiment, the photoactivating light is ultraviolet (UV) light. In one embodiment, the photoactivating light has a wavelength within the range of about 300 to about 500 nm.

In one embodiment, the photoactivating light is laser radiation. In one embodiment, the photoactivating light is produced by a light emitting diode (LED) device.

In one embodiment, the photoactivating light source is an external source of electromagnetic radiation. In one embodiment, the photoactivating light source is external and a waveguide (e.g., optical fiber) is employed for the delivery of the emitted light to the intervention site. In one embodiment, the photoactivating light source is provided by using designated over-the wire delivery system. As used herein, an over the wire delivery system comprises a delivery catheter which can be utilized with a guide wire to provide an over-the-wire delivery device to the intervention site in a blood vessel. In one embodiment, the photoactivating light source is detachable from the catheter and the delivery system is withdrawn from the intervention site after the light source is detached. In one embodiment, the delivery catheter is withdrawn, leaving the guide wire attached to the photoactivating light source at the intervention site. In one embodiment, the external source of electromagnetic radiation is a vascular catheter comprising a LED device at one end of the catheter.

In one embodiment, the cross-linking agent is released in a therapeutically effective amount at the intervention site (in-situ). In one embodiment, the release of a therapeutically effective amount of the cross-linking agent occurs within 1 second and 40 seconds from its placement at the intervention site. In one embodiment, within 30 seconds post initiating expansion (e.g., inflation) of the expandable member more than 80% of cross-linking agent is released from the expandable member surface into the surface of an intervention site. In some embodiments, 80% or more of the cross-linking agent is released between 20 and 40 seconds after having initiated expansion of the expandable member.

In one embodiment, photoactivation occurs immediately with the release of the cross-linking agent at the intervention site.

In one embodiment, the device comprises a tubular vascular implant.

In one embodiment, the present invention provides a method of delivering a cross-linking agent to a vessel wall of a body lumen, wherein the vessel wall comprises collagen. The method comprises providing a device according to embodiments of the invention. The method further comprises positioning the expandable member in an operable position at an intervention site within a body lumen; expanding the expandable member to engage with the intervention site within the vessel wall; intraluminally releasing, the cross-linking agent to the vessel wall; and photoactivating the cross-linking agent for cross-linking of collagen surrounding the expandable member.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1 is a schematic view of an exemplary implementation of a device for treating and/or preventing restenosis in an intervention site, in accordance with an embodiment;

FIG. 2 is a schematic view of an exemplary implementation of the device of FIG. 1, in accordance with another embodiment;

FIG. 3 is a schematic view of an exemplary implementation of the device of FIG. 1, in accordance with another embodiment;

FIG. 4 is a schematic view of an exemplary implementation of the device of FIG. 1, in accordance with another embodiment;

FIG. 5 is a schematic view of an exemplary implementation of the device of FIG. 1, in accordance with another embodiment;

FIG. 6 is a schematic view of an exemplary implementation of the device of FIG. 1, in accordance with another embodiment;

FIG. 7 is a schematic view of an exemplary implementation of the device of FIG. 1, in accordance with another embodiment;

FIG. 8 is a schematic view of an exemplary implementation of the device of FIG. 1, in accordance with another embodiment;

FIG. 9 is a schematic view of an exemplary implementation of a system including the device of FIG. 1, in accordance with another embodiment; and

FIG. 10 is a flow chart of a method utilizing the devices and/or a system of FIGS. 1-9 for treating and/or preventing restenosis.

### DETAILED DESCRIPTION

The present invention provides devices, systems and methods useful for treating and/or preventing restenosis. According to an embodiment, restenosis is treated and/or prevented by changing a property of the extracellular matrix (ECM) within a tissue surrounding a vascular stent or atherosclerotic lesion. According to an embodiment, treating, decreasing, ameliorating, and/or preventing restenosis is achieved by cross linking a collagen fibril of the ECM. According to an embodiment, cross linking is induced (e.g., catalyzed) by a photosensitizer agent which is photoactivated by a light source in order to cross-link collagen of the ECM. In one embodiment, cross linking a collagen fibril of the extracellular matrix decrease the risk of distal embolization using stent delivery or angioplasty.

It is noted that the terms “collagen” and “collagen fibril” as used herein can be used interchangeably.

Throughout the text the terms “vessel” and “blood vessel” are used interchangeably and refer to a blood vessel of interest for the intervention procedure. In one embodiment, the blood vessel is a coronary blood vessel. In one embodiment, the blood vessel is an artery. In one embodiment, the blood vessel is a coronary artery. In one embodiment, the blood vessel is a blood vessel in proximity to a coronary artery. In one embodiment, the blood vessel is a peripheral blood vessel. In one embodiment, the blood vessel is a renal artery. In one embodiment, the blood vessel is a venous graft. In one embodiment, a blood vessel is an arterial graft. In one embodiment, a blood vessel is a vein. In one embodiment, the intervention site is the site wherein the intervention procedure for the prevention and/or treatment of restenosis is carried out.

It should be noted that the term “light” as used herein may refer to electromagnetic radiation of any suitable wavelength for the purposes of the applications disclosed herein. Accordingly, the term “light” should not be construed as being limited to visible light and may additionally or alternatively include non-visible radiation such as, for example, laser light in the infrared range and UV light.

Disclosed herein is a device comprising: an expandable member having a distal end, a proximal end and a working length there between; an active agent (e.g., a cross-linking agent); and a photoactivating light source operative to emit light for activating, when in an operable position, the active agent. Optionally, when the expandable member and the photoactivating light source are in operable position, the cross-linking agent cross-links or catalyzes the cross linking of a collagen surrounding the expandable member. In some embodiments, there is provided a system comprising a device as described herein and a power source for actuating the photoactivating light source and/or the expansion (e.g., inflation) of the expandable member.

Optionally, the device comprises a cross linking agent-eluting expandable member, wherein the expandable member includes the photoactivating light source. Optionally, the device comprises the cross linking agent-eluting expandable member, wherein the photoactivating light source may be embedded within the expandable member. In one embodiment, the device comprises a cross linking agent-eluting balloon, wherein the photoactivating light source may be placed on an outer surface of the expandable/inflatable balloon. Optionally, the photoactivating light source may be placed on at least a portion of an outer surface of the inflatable balloon. It is noted that the terms “inflatable” and “expandable” as used herein can be used interchangeably.

Optionally, the expandable member is a balloon. Optionally, the expandable member is made of a deformable and/or an expandable latex, plastic or rubber.

The expandable member (e.g., balloon) may be made of a wide variety of materials including, for example, polytetrafluoroethylenes (Teflon®), polyethylenes, (e.g., high density polyethylenes), polyethylene terephthalate (PET), polypropylenes, polyurethanes, nylons including nylon 6 and nylon 12, polyesters including polyalkylene terephthalate polymers and copolymers, (e.g., thermoplastic polyester elastomers such as Hytrel®, which is a block copolymer containing a hard polybutylene terephthalate segment and soft amorphous segments based on long-chain polyether glycols), polyimides, polyamides including polyether-block-co-polyamide polymers (e.g., Pebax®), and the like. These materials may also be blended or provided in a composite or multi-layer construction. Typically, PET balloons are substantially optically clear and permit the transmission of light over a broad spectrum.

Optionally, the expandable member size ranges from 2 to 25 millimeter (mm) in diameter, or alternatively 4 to 12 mm in diameter, or alternatively 2 to 4 mm in diameter. Optionally, the expandable member size ranges from 8 to 40 mm in length, or alternatively 20 to 100 mm in length. Optionally, the expandable member is rated for a pressure capability of 5 to 20 atmospheres (ATM), or alternatively 8 to 20 ATM, or alternatively 10 to 20 ATM or alternatively 5 to 15 ATM. Each possibility represents a separate embodiment of the present invention. The different embodiments can be combined at will.

Optionally, the active agent (e.g., cross-linking agent) is a photoactivatable substance. Optionally, the photoactivating light source may activate the active agent in situ. Alternatively, the photoactivating light source may activate the active agent within a blood vessel and/or a vascular tissue. Alternatively, the photoactivating light source may activate the active agent upon release within a blood vessel.

In a non-limiting example, the active agent is a cross-linking agent. In a non-limiting example, the cross-linking agent is capable of cross linking an organic molecule. In a non-limiting example, the cross-linking agent is capable of cross linking a protein. In a non-limiting example, the cross-linking agent is capable of cross linking a structural protein. In a non-limiting example, the cross-linking agent is capable of cross linking a connective tissue structural protein. Alternatively, the cross-linking agent is a photosenitizer. Typically, the photosenitizer doesn't form a chemical interaction with the ECM. The term “photosensitizer” as used herein, refers to an agent that is capable of absorbing light energy (e.g., from the photoactivating light source) and deliver, as a result of the light absorption, energy to catalyze a chemical reaction.

In a non-limiting example, the cross-linking agent may include one or more photoactive bioflavonoids selected from the group consisting of proanthocyanidin, catechin, epicatechin, epigallo catechin, epicatechin gallate, epigallocatechin gallate, quercetin, tannic acid, and any combination thereof. In another non-limiting example, the cross-linking agent comprises one or more photoactive substances. In yet another non-limiting example, the cross-linking agent comprises a photoactive substance that comprises riboflavin.

In another non-limiting example, the cross-linking agent comprises an enzyme capable of cross-linking collagen fibers. In a non-limiting example, lysyl oxidase is used to cross-link collagen fibers. In another non-limiting example, the cross-linking agent comprises: carbodiimide, polyepoxy ethers, divinyl sulfone (DVS), genipin, polyaldehyde and diphenylphosphoryl azide (DPPA) or combinations thereof.

Optionally, the device further comprises an agent capable of preventing collagen degradation. In a non-limiting example, the agent capable of preventing collagen degradation is a metalloproteinase inhibitor. Optionally, the agent capable of preventing collagen degradation (e.g., metalloproteinase inhibitor) is released along with the cross-linking agent.

Optionally, the cross-linking agent may induce cross-linking of a collagen fibril at the intervention site, following a photoactivating light incident on the cross-linking agent. Optionally, the photoactivating light initiates the cross-linking activity by irradiating the applied cross-linking agent (via the release of reactive oxygen radicals). Optionally, the cross-linking agent acts as a sensitizer to convert O₂ into singlet oxygen which causes cross-linking of collagen within a biological tissue. In a non-limiting example, the biological tissue is a biological tissue comprising of collagen fibrils selected from: a venous tissue, a cardiac valvular tissue, a connective tissue, a vascular tissue, a cutaneous or subcutaneous tissue, a tissue of a muscular tendon, a tissue of a muscular fascia a tissue of a muscular aponeurosis or an extracellular matrix tissue. In one embodiment, the biological tissue is the extracellular matrix.

Optionally, the device further includes a “carrying agent”. As used herein, the carrying agent is any mixture of inert materials which will deliver or release the active agent, the metalloproteinase inhibitor and/or the cross linking agent in situ and in a desired elution profile. Optionally, the carrying agent provides a predefined release profile of the cross linking agent. Optionally, the carrying agent glues or adheres the active agent, the metalloproteinase inhibitor and/or cross linking agent to the expandable member. In a non-limiting example, carrying agent is a polymer. In one embodiment, carrying agent is a resin. In another non-limiting example, the carrying agent is biologically inert.

Optionally, the photoactivating light is ultraviolet (UV) light. In one embodiment, the photoactivating light has a wavelength within the range of 10 to 500 nm, 10 to 400 nm, or 300 to 500 nm. Each possibility represents a separate embodiment of the present application. The different embodiments can be combined at will. In one embodiment, the photoactivating light has a wavelength within the range of 450 to 480 nm.

Optionally, the photoactivating light is a radiation produced by a laser device. In one embodiment, the photoactivating light is a radiation produced by a light emitting diode (LED) device. Optionally, the photoactivating light source is an external source of electromagnetic radiation. In one embodiment, the external source of electromagnetic radiation is a vascular catheter with LED device operatively coupled thereto. Optionally, the external source of electromagnetic radiation is a vascular catheter operative to allow also the passage of an optical fiber able to convey a laser radiation.

Optionally, the photoactivating light source can be embedded in the distal end, proximal end or working length of the expandable member. In one embodiment, the photoactivating light source is located within the expandable member. Optionally, the photoactivating light source covers at least a portion of the expandable member. In one embodiment, the LED is located within the expandable member.

Optionally, the device is a tubular vascular implant. Optionally, the expandable member acquires an expanded configuration at the intervention site only.

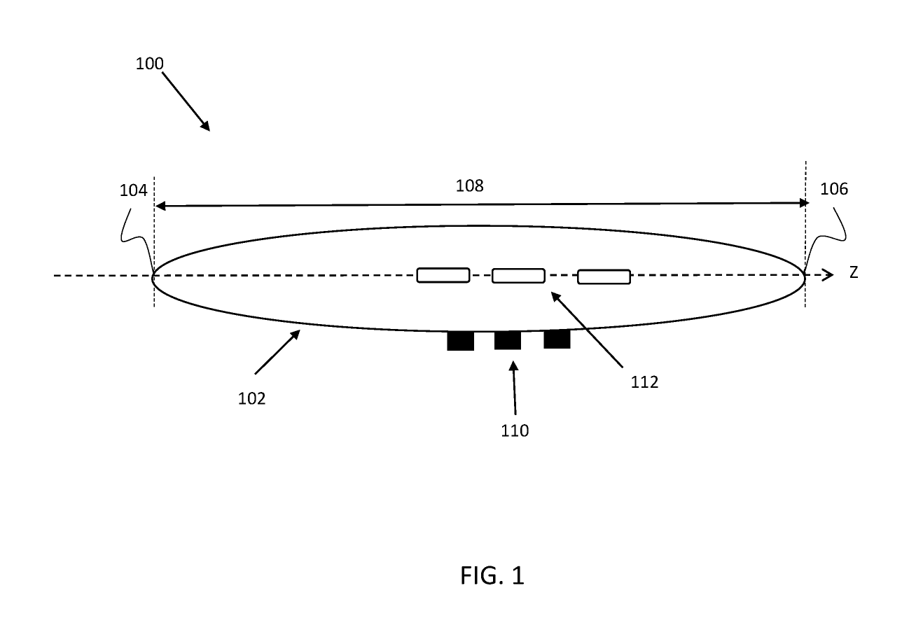

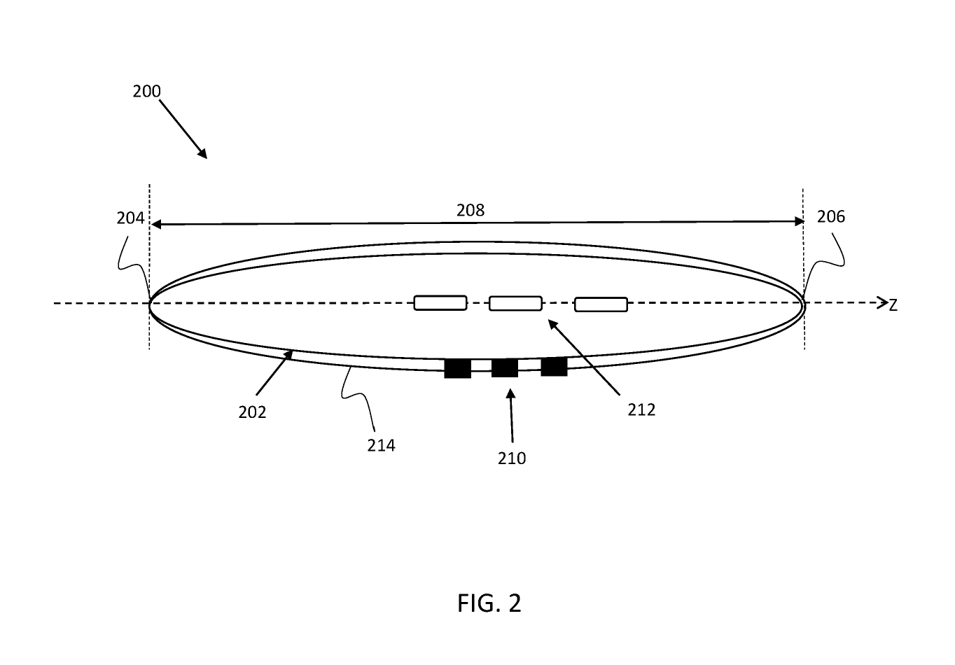

Throughout the following description, similar elements of different embodiments of the device are referenced by element numbers differing by integer multiples of 100. For example, expandable member of FIG. 1 is referenced by the number 102, and expandable member of FIG. 2, which corresponds to expandable member 102, is referenced by the number 202.

Reference is now made to FIG. 1 which shows a device 100 that may be used for treating or preventing restenosis in an intervention site. Device 100 includes an expandable member (e.g. balloon) 102 having a proximal end 104, a distal end 106 and a working length 108 there between; an active agent 110 (e.g., a cross-linking agent); and a photoactivating light source 112 operative to emit light for activating the active agent.

Optionally, active agent 110 overlays, at least partially, expandable member 102. Optionally, photoactivating light source 112 is located within expandable member 102, such as on a central longitudinal axis Z that runs along a length of expandable member 102 from proximal end 104 to distal end 106.

Reference is now made to FIG. 2 which shows a device 200, in accordance with another embodiment. Device 200 is substantially similar to device 100 described in FIG. 1A with the notable difference that an expandable member 202, of device 200 is further coated with a layer 214. Optionally, layer 214 is also expandable upon expansion of expandable member 202. Optionally, an active agent 210 is embedded in layer 214 coating expandable member 202. Optionally, layer 214 comprises a “carrying agent”. Optionally, layer 214 comprises the carrying agent and active agent 210.

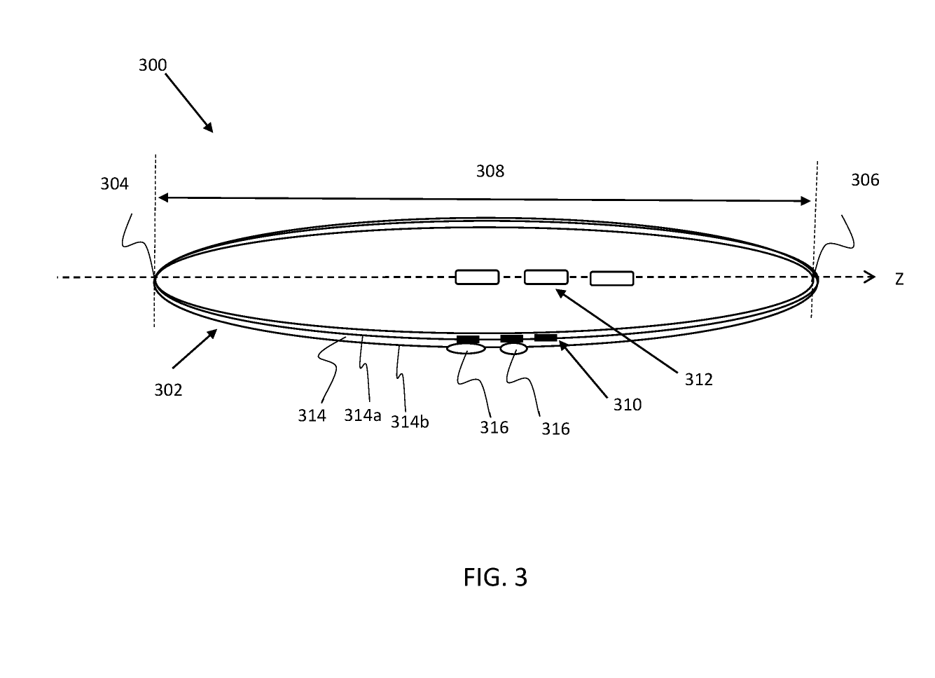

Reference is now made to FIG. 3 which shows a device 300, in accordance with another embodiment. Device 300 is substantially similar to device 100 described in FIG. 1 with the notable difference that an expandable member 302 is coated by multi-layers 314. Optionally, multi layers 314 include at least two layers a first layer 314a and a second layer 314b. Optionally, first layer 314a includes an active agent 310. Optionally, second layer 314b includes one or more release sites or ports 316 for the elution of an active agent 312. Optionally, release sites 316 may be embodied by holes (e.g., pores). Optionally, at least two of release sites 316 may differ in size from one another. Optionally, a wall thickness of expandable member 302 may be non-uniform. For example, the wall thickness at a proximal end 304 and a distal end 306 of expandable member 302 may differ from the wall thickness along a working length 308 of expandable member 302. In a further example, a wall thickness at proximal end 304, distal end 306 and along working length 308 of expandable member 302, may differ from each other. Additional or alternative configurations are possible.

For a non-limiting example, expandable member 302 may be formed, at least partially, of a porous material. In another non-limiting example, expandable member 302 may be formed, at least partially, of a porous material which provides release sites 316 and is formed therein one or more release sites 316. Optionally, active agent 312 is embedded in layers 314 coating expandable member 302. Optionally, active agent 312 is embedded in a section of layers 314 coating working length 308 of expandable member 302. Optionally, release sites 316 are arranged along a portion of working length 308, such that active agent 310 is released through release sites 316. Optionally, release sites 316 of expandable member 302 are in the form of reservoirs containing active agent 310 for release.

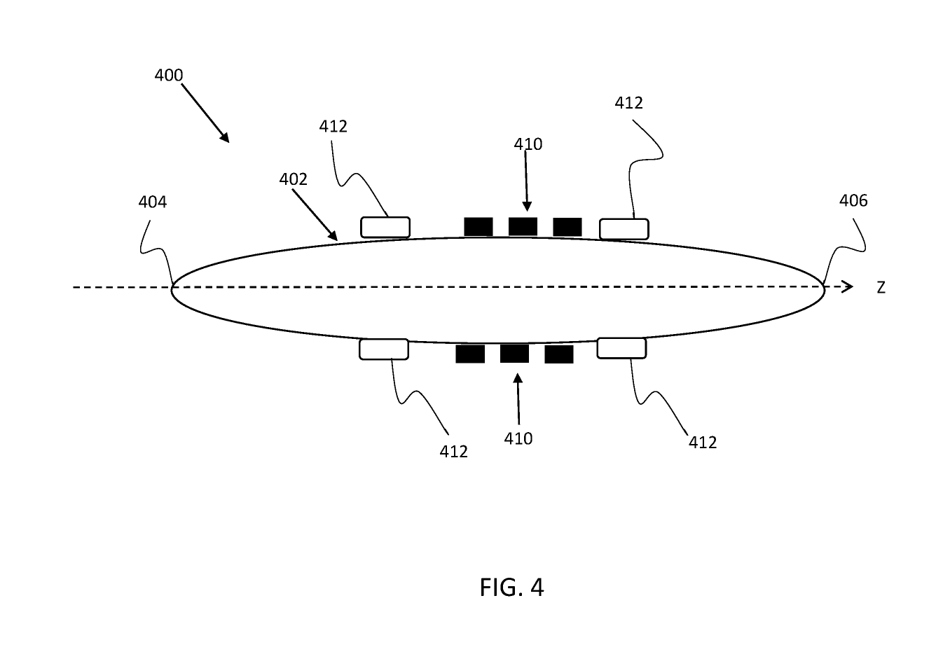

Reference is now made to FIG. 4 which shows a device 400, in accordance with another embodiment. Device 400 is substantially similar to device 100 described in FIG. 1 with the notable difference that a photoactivating light source 412 is adhered onto a surface of an expandable member 402.

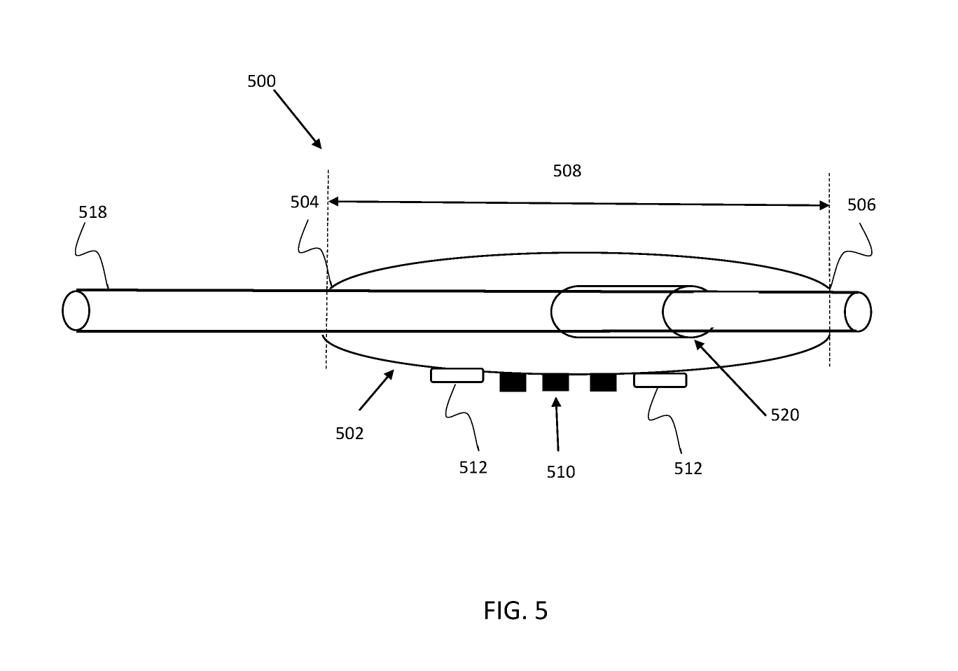

Reference is now made to FIG. 5 which shows a device 500, in accordance with another embodiment. Device 500 is substantially similar to device 400 described in FIG. 4 with the notable difference that device 500 further includes a catheter 518. Optionally, catheter 518 is inserted through a lumen of the expandable member 502. Optionally, catheter 518 includes at least one tube-shaped or hollow elongate body 520. Optionally, a fluid for expanding expandable member 502 may be supplied through at least one tube-shaped or hollow elongate body 520.

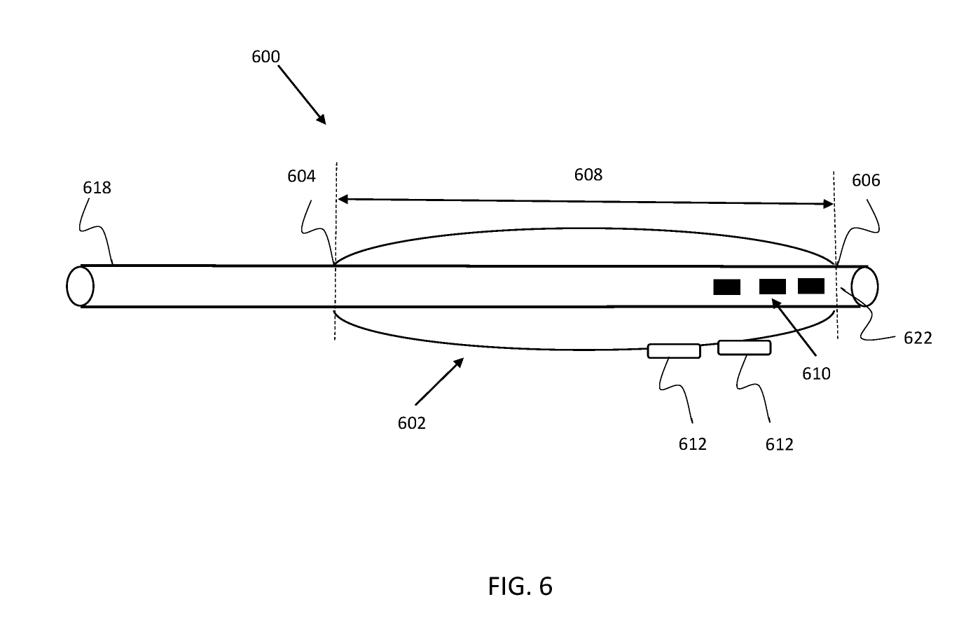

Reference is now made to FIG. 6 which shows a device 600, in accordance with another embodiment. Device 600 is substantially similar to device 500 described in FIG. 5 with the notable difference that catheter 618 is operable to supply an active agent 610 to the intervention site. Optionally, the catheter 618 is operable to supply an active agent 610 and a carrying agent through catheter 618. Optionally, catheter 618 includes a lumen 622 through which active agent 610 may be supplied.

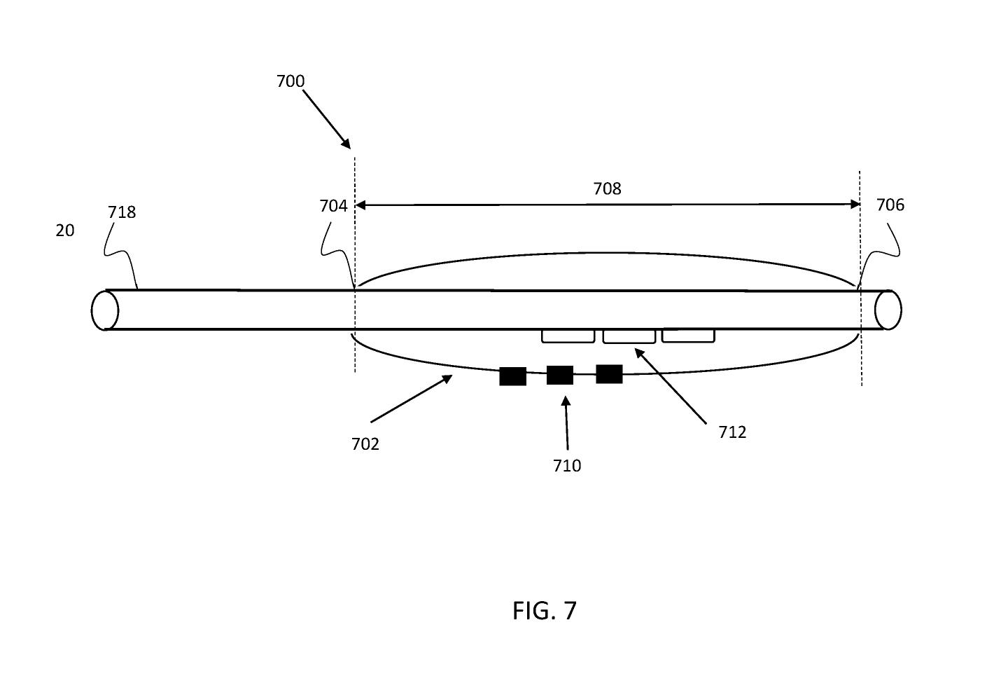

Reference is now made to FIG. 7 which shows a device 700, in accordance with another embodiment. Device 700 is substantially similar to device 500 described in FIG. 5 with the notable difference that a catheter 718 is operable to provide a photoactivating light source 712, e.g., to the intervention site. Optionally, photoactivating light source 712 is coupled with catheter 718. Optionally, the catheter 718 includes photoactivating light source 712 at one end (distal or proximal).

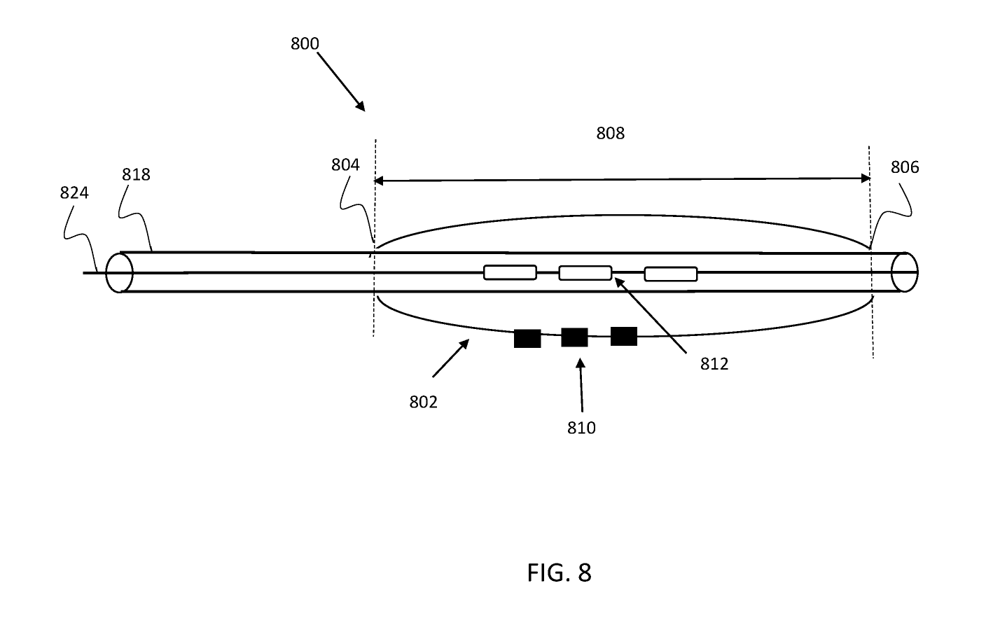

Reference is now made to FIG. 8 which shows a device 800, in accordance with another embodiment. Device 800 is substantially similar to device 700 described in FIG. 7 with the notable difference that device 800 further includes a guide-wire 824. Optionally, a photoactivating light source 812 is embedded in or arranged on guide-wire 824. Optionally, a catheter 818 is operative to allow the passage of a waveguide to act as a source that provides photoactivating light to the intervention site.

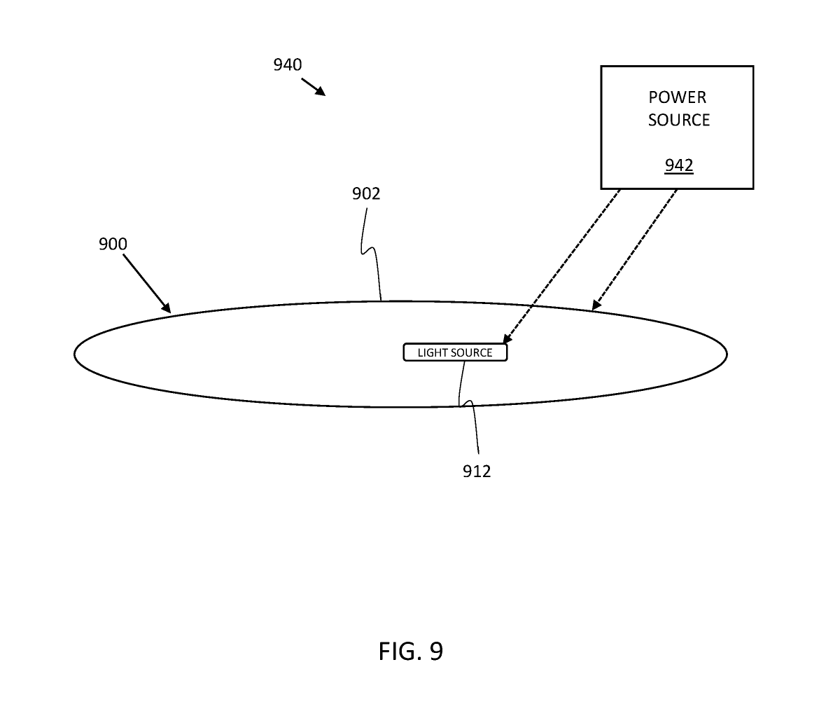

Reference is now made to FIG. 9 which shows a system 940 that may be used for treating and/or preventing restenosis. System 940 includes any of the devices described in FIGS. 1-8 and a power source 942. Power source 942 may be utilized for actuating a photoactivating light source 912 and/or expansion (e.g., inflation) of an expandable member 902.

According to another aspect, the invention provides a method for treating and/or preventing restenosis. Optionally, the invention provides a method of delivering a cross-linking agent to a vessel wall of body lumen (such as of a blood vessel), wherein the vessel wall comprises collagen.

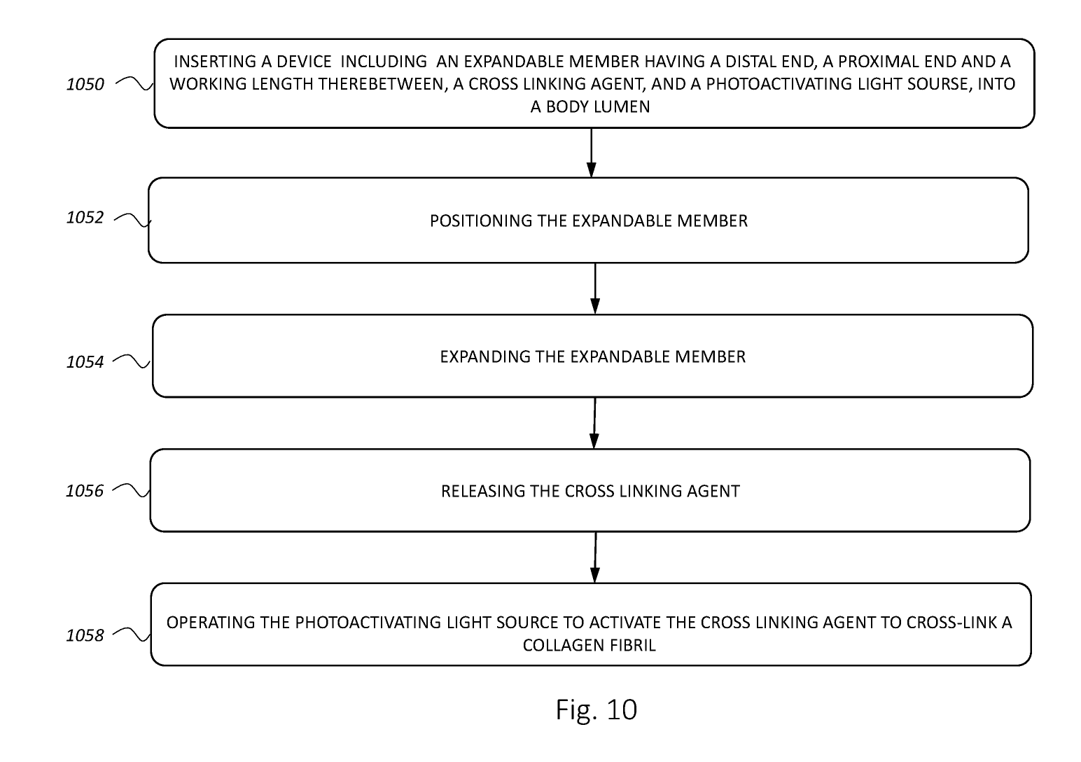

Reference is now made to FIG. 10, which is a flow chart of the method for treating and/or preventing restenosis in an intervention site, in accordance with the devices and system of FIGS. 1-9. The device and/or system is inserted, in its collapsed configuration, into a body lumen (a blood vessel) (Step 1050). Optionally, the device employs a catheter to be inserted into a vessel.

An expandable member of the device is positioned within an intervention site of a body lumen (such as of a blood vessel) (Step 1052). The expandable member is expended (Step 1054). Optionally, an expandable member surrounds a portion of the catheter near or at a distal end of the catheter and the catheter is operatively coupled with an expansion mechanism for expanding the expandable member. For example, the catheter may comprise a tube operative to provide inflation fluid for inflating a balloon. In an alternative embodiment, the expandable member/inflatable balloon releases the catheter at the intervention site and for expanding (e.g., through inflation) the expandable member to a predetermined size.

The cross-linking agent is released intraluminally on to the vessel wall (Step 1056). Optionally, the cross-linking agent is released in a therapeutically effective amount at an intervention site. Optionally, the cross-linking agent is released in a therapeutically effective amount upon inflation/expansion of the expandable member. Optionally, the release of a therapeutically effective amount of cross-linking chemical occurs in between 1 second and 40 seconds. Optionally, the release occurs in between 1 second and 20 seconds. Optionally, the release occurs in between 1 second and 10 seconds. Optionally, the intervention site is within a blood vessel. Optionally, the release of a therapeutically effective amount of cross-linking chemical is triggered by expansion of the expandable member into an expanded configuration. Optionally, 30 seconds post initial expansion (e.g., inflation) of the expandable member, more than 80% of cross-linking agent is released from the expandable member surface into the surface of the intervention site. Optionally, more than 40%, more than 50%, more than 60%, or more than 70% of the cross-linking agent is released from the expandable member surface within 30 seconds from expansion of the expandable member. Alternatively, the cross linking agent coating the expandable member may be pressed against the vessel wall upon expansion of the expandable member.

The photoactivating light source is operated to photoactivate the cross-linking agent, thus cross-linking a collagen fibril (Step 1058).

Optionally, the photoactivating light source is supplied by a catheter, wherein the catheter is inserted into a vessel. Alternatively, the light source may be supplied close to a vessel wall, e.g., by the cavity of the expandable member.

Optionally, irradiation for photoactivation occurs immediately with the release of the cross-linking agent at the intervention site. Optionally, irradiation for photoactivation is concomitantly performed while expanding of the expandable member into expanded configuration. Optionally, photoactivation by the light source is effected responsive to irradiating the cross-linking agent with a single pulse having a duration of, e.g., 4 to 60 seconds, 4 to 20 seconds, 20 to 60 seconds, or 10 to 30 seconds. Optionally, photoactivation by the light source is effected responsive to irradiating the cross-linking agent with a series of multiple pulses over a duration of, e.g., 4 to 60 seconds, 4 to 20 seconds, 20 to 60 seconds, or 10 to 30 seconds. Optionally, each photoactivation pulse lasts from e.g., 1 Pico second to 5 seconds, from 1 Pico second to 1 millisecond, from 1 millisecond second to 1 second, or from 1 second to 10 seconds. Each possibility represent a separate embodiment of the present invention.

Optionally, the activated cross-linking agent is used for cross-linking a collagen fibril. In one embodiment, collagen is any one of type I to XXVIII collagen. In one embodiment, collagen fibrils compose part of the extracellular matrix (ECM). Optionally, cross-linking collagen fibrils change the permeability of the ECM.

Optionally, after using the device to cross-link collagen fibrils of the ECM, the device is withdrawn and a vascular (e.g., BMS or DES) stent may be delivered.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended.

Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art.

1. A device comprising:
an expandable member having a distal end, a proximal end and a working length therebetween;
a cross-linking agent; and
a photoactivating light source.

2. The device of claim 1, wherein the expandable member is a balloon.

3. The device of claim 1, wherein the expandable member is coated with a layer comprising a carrying agent.

4-5. (canceled)

6. The device of claim 1, wherein the cross-linking agent is supplied through a catheter of the device.

7. The device of claim 1, wherein the photoactivating light source is supplied by and/or incorporated on the expandable member.

8-9. (canceled)

10. The device of claim 1, wherein in operable position, the cross-linking agent induces cross-linking of collagen fibrils responsive to photoactivating light incident on the cross-linking agent.

11. The device of claim 1, wherein the cross linking agent comprises one or more photoactive bioflavonoids selected from the group consisting of: proanthocyanidin, catechin, epicatechin, epigallo catechin, epicatechin gallate, epigallocatechin gallate, quercetin, tannic acid, and any combination thereof.

12. The device of claim 1, wherein the cross-linking agent comprises one or more photoactive substances.

13. The device of claim 10, wherein the cross-linking agent comprises riboflavin.

14-15. (canceled)

16. The device of claim 1, wherein the photoactivating light is ultraviolet (UV) light.

17. The device of claim 1, wherein the photoactivating light has a wavelength within the range of 300-500 nm.

18. (canceled)

19. The device of claim 1, wherein the photoactivating light is produced by a LED device.

20. The device of claim 1, wherein the source is an external source of electromagnetic radiation.

21. The device of claim 1, wherein the photoactivating light source is external and uses a waveguide for its delivery.

22. (canceled)

23. The device of claim 1, wherein the photoactivating light source is a vascular catheter comprising a LED device.

24. The device of claim 1, wherein the photoactivating light source is a vascular catheter operative to allow the passage of an optical fiber able to convey a laser radiation.

25. (canceled)

26. The device of claim 1, wherein the cross-linking agent is released in a therapeutically effective amount at an intervention site (in-situ).

27. (canceled)

28. The device of claim 26, wherein 30 seconds post initial expansion of the expandable member more than 80% of cross-linking agent is released from the expandable member surface into the surface of the intervention site.

29-30. (canceled)

31. A method of delivering a cross-linking composition to a vessel wall of a body lumen, wherein the vessel wall comprises collagen, comprising the steps of:
providing an expandable member having a distal end, a proximal end and a working length therebetween; cross-linking agent; and a photoactivating light source;
positioning the expandable member within an intervention site of a body lumen;
expanding the expandable member and releasing intraluminally the cross-linking agent onto the vessel wall; and
photoactivating the cross-linking agent, for cross-linking a collagen fibrils of the vessel wall.
